SEQUENCE LISTING (13 sequences)

SEQ ID NO: 1 - protein, 124 aa, Artificial Sequence
  Description of Artificial Sequence Synthetic polypeptide
GCIEEQAKTFLDKFNHEAEDLFYQSSLASWNYNTNITEENVQNMNNAGDKWSAFLKEQSTLAQMYACGGGNLVTAFSNMDDMLQKAHLVIEGTFIYLRDSTEFFIRVRDGWKKLQLGELIPIPA

SEQ ID NO: 2 - protein, 130 aa, Artificial Sequence
  Description of Artificial Sequence Synthetic polypeptide
STIEEQAKTFLDKFNHEAEDLFYQSSLASWNYNTQITEENVQNKNNAGDKKSAFLKEQSTLAQMYPLQEIQNLGGGNLVTAFSNMDDMLQKAHLVIEGTFIYLRDSTEFFIRVRDGWKKLQLGELIPIPA

SEQ ID NO: 3 - protein, 130 aa, Artificial Sequence
  Description of Artificial Sequence Synthetic polypeptide
STIEEQAKTFLDKFNHEAEDLFYQSSLASWNYNTQITEENVQNRNNAGDKRSAFLKEQSTLAQMYPLQEIQNLGGGNLVTAFSNMDDMLQKAHLVIEGTFIYLRDSTEFFIRVRDGWKKLQLGELIPIPA

SEQ ID NO: 4 - protein, 327 aa, Artificial Sequence
  Description of Artificial Sequence Synthetic polypeptide
QPAVVHLQGQGSAIQVKNDLSGGVLNDWSRITMNPKVFKLHPRSGELEVLVDGTYFIYSQVEVYYINFTDFASYEVVVDEKPFLQCTRSIETGKTNYNTCYTAGVCLLKARQKIAVKMVHADISINMSKHTTFFGAIRLGEAPAPGAVVCVESEIRGDVTIGPRTVIHPKARIIAEAGPIVIGEGNLIEEQALIINAYPDNIKPMIIGTNNVFEVGCYSQAMKMGDNNVIESKAYVGRNVILTSGCIIGACCNLNTFEVIPSTIEEQAKTFLDKFNHEAEDLFYQSSLASWNYNTNITEENVQNMNNAGDKWSAFLKEQSTLAQMYG

SEQ ID NO: 5 - protein, 614 aa, Artificial Sequence
  Description of Artificial Sequence Synthetic polypeptide
STIEEQAKTFLDKFNHEAEDLFYQSSLASWNYNTNITEENVQNMNNAGDKWSAFLKEQSTLAQMYGGYPDNIKPMIIGTNNVFEVGCYSQAMKMGDNNVIESKAYVGRNVILTSGCIIGACCNLNTFEVIPENRTVIHPKARIIAEAGPIVIGEGNLIEEQALIINAYPDNIKPMIIGTNNVFEVGCYSQAMKMGDNNVIESKAYVGRNVILTSGCIIGACCNLNTFEVIPENRTVIHPKARIIAEAGPIVIGEGNLIEEQALIINAYPDNIKPMIIGTNNVFEVGCYSQAMKMGDNNVIESKAYVGRNVILTSGCIIGACCNLNTFEVIPENRTVIHPKARIIAEAGPIVIGEGNLIEEQALIINAYPDNIKPMIIGTNNVFEVGCYSQAMKMGDNNVIESKAYVGRNVILTSGCIIGACCNLNTFEVIPENRTVIHPKARIIAEAGPIVIGEGNLIEEQALIINAYPDNQPAVVHLQGQGSAIQVKNDLSGGVLNDWSRITMNPKVFKLHPRSGELEVLVDGTYFIYSQVEVYYINFTDFASYEVVVDEKPFLQCTRSIETGKTNYNTCYTAGVCLLKARQKIAVKMVHADISINMSKHTTFFGAIRLGEAP

SEQ ID NO: 6 - protein, 66 aa, Artificial Sequence
  Description of Artificial Sequence Synthetic polypeptide
STIEEQAKTFLDKFNHEAEDLFYQSSLASWNYNTNITEENVQNMNNAGDKWSAFLKEQSTLAQMYP

SEQ ID NO: 7 - protein, 74 aa, Artificial Sequence
  Description of Artificial Sequence Synthetic polypeptide
STIEEQAKTFLDKFNHEAEDLFYQSSLASWNYNTNITEENVQNMNNAGDKWSAFLKEQSTLAQMYPLQEIQNLG

SEQ ID NO: 8 - protein, 118 aa, Artificial Sequence
  Description of Artificial Sequence Synthetic polypeptide
GAVVCVESEIRGDVTIGPRTVIHPKARIIAEAGPIVIGEGNLIEEQALIINAYPDNIKPMIIGTNNVFEVGCYSQAMKMGDNNVIESKAYVGRNVILTSGCIIGACCNLNTFEVIPEP

SEQ ID NO: 9 - protein, 54 aa, Artificial Sequence
  Description of Artificial Sequence Synthetic polypeptide
NLVTAFSNMDDMLQKAHLVIEGTFIYLRDSTEFFIRVRDGWKKLQLGELIPIPA

SEQ ID NO: 10 - protein, 163 aa, Artificial Sequence
  Description of Artificial Sequence Synthetic polypeptide
GSHMGPSGAADKAGTRENQPAVVHLQGQGSAIQVKNDLSGGVLNDWSRITMNPKVFKLHPRSGELEVLVDGTYFIYSQVEVYYINFTDFASYEVVVDEKPFLQCTRSIETGKTNYNTCYTAGVCLLKARQKIAVKMVHADISINMSKHTTFFGAIRLGEAPAS

SEQ ID NO: 11 - protein, 182 aa, Artificial Sequence
  Description of Artificial Sequence Synthetic polypeptide
ASTLNAQIPELKSDLEKASALNTKIRALQGSLENMSKLLKRQNDILQVVSQGWKYFKGNFYYFSLIPKTWYSAEQFCVSRNSHLTSVTSESEQEFLYKTAGGLIYWIGLTKAGMEGDWSWVDDTPFNKVQSARFWIPGEPNNAGNNEHCGNIKAPSLQAWNDAPCDKTFLFICKRPYVPSEP

SEQ ID NO: 12 - protein, 76 aa, Artificial Sequence [sequence omitted]

SEQ ID NO: 13 - protein, 15 aa, Artificial Sequence
  Description of Artificial Sequence Synthetic peptide
ADDCLRRVQTERPQP

(19) United States
(12) Patent Application Publication
(10) Pub. No.: US 2023/0382977 A1
(43) Pub. Date: Nov. 30, 2023

(54) MULTIMERIC PROTEIN COMPLEXES AS ANTIBODY SUBSTITUTES FOR NEUTRALIZATION OF VIRAL PATHOGENS IN PROPHYLACTIC AND THERAPEUTIC APPLICATIONS

(71) Applicants: The Regents of the University of California, Oakland, CA (US); Protein Architects Corporation, Middletown, DE (US)

(72) Inventors: Daniel L. COX, San Francisco, CA (US); Richard L. DAVIS, Penn Valley, PA (US); Michael D. TONEY, Davis, CA (US)

(21) Appl. No.: 18/028,345

(22) PCT Filed: Sep. 23, 2021
(86) PCT No.: PCT/US2021/051772

§ 371 Date: Mar. 24, 2023

Related U.S. Application Data
(60) Provisional application No. 63/082,587, filed on Sep. 24, 2020.

(51) Int. Cl. C07K 16/10

(52) U.S. Cl. CPC C07K 16/1003; C07K 2318/20; C07K 2317/76; C07K 2319/31; C07K 2317/92; C07K 2319/735

(57) ABSTRACT

The present patent consists of an engineered multimeric protein complex as antibody substitute composed of human proteins, with an m-fold symmetry, with each m-fold element containing a modified monomeric protein derived from a symmetric human multimeric protein complex fused to a module containing n fused, modified human beta solenoid proteins (mBSP), and that fused to a human derived pathogen binding domain (PBD), as well as a separate antibody substitute composed of P human PBD complexes. The invention may find application in prophylactic and therapeutic treatments for viral infections, especially for COVID19 by neutralizing the SARS-CoV-2 virus.

### CROSS-REFERENCE TO RELATED PATENT APPLICATIONS

The present patent application is a US National Phase Application Under 371 of International Application PCT/US2021/051772 filed Sep. 23, 2021, which claims benefit of priority to U.S. Provisional Patent Application No. 63/082,587, filed Sep. 24, 2020, each of which is incorporated by reference for all purposes.

### SEQUENCE LISTING

The instant application contains a Sequence Listing which has been submitted electronically in ASCII format and is hereby incorporated by reference in its entirety. Said ASCII copy, created on Oct. 22, 2021, is named 081906-1271535-240910PC_SL.txt and is 20,094 bytes in size.

### BACKGROUND OF THE INVENTION

Even when effective vaccines for COVID19 become available for broad application, there will remain a need for prophylactic and/or therapeutic inventions that will inhibit infection by SARS-CoV-2 and its mutational descendants. This need is especially acute for urgent care providers, community police, and military personnel in close quarters for extended periods of time. Frequent hand washing, wearing of the accepted standard of face masks, and physical distancing are proven to be effective for the general population, but are not practical for these essential sub-populations. Monoclonal antibody-based prophylactics are potentially highly effective, but the cost of production from mammalian cells limits applicability and large scale social impact. This is also true of monoclonal antibody-based intravenous therapeutics. Additionally, monoclonal antibodies are relatively unstable environmentally, not easily adapted to viral mutational descendants, and risk triggering unwanted viral responses such as antibody dependent enhancement (ADE) of infection or autoimmune responses such as the cytokine storm observed in some COVID19 patients. The inventions described here offer an alternative to the monoclonal antibody standard. They are a novel extension of previously proven out-of-body antiviral biotechnology: programmatic development of human body-friendly synthetic antibody substitutes that are mass producible at low cost, highly adaptable to emerging zoonotic viral threats, and applicable to COVID19 and future viral threats.

The inventions disclosed can be utilized as an alternative to any monoclonal antibody. Of the top ten biotechnology drugs in 2014 according to Statista, three where monoclonal antibodies to Tumor Necrosis Factor (TNF)—Humira/adalimumab, Remicade/infliximab, Enbrel/etanercept, followed by three other monoclonal antibodies: Rituxan/rituximab (anti-CD20 mAb), Avastin/bevacizumab (anti-VEGF mAb) and Herceptin/trastuzmab (anti-HER2 mAb). These six monoclonal antibodies had aggregate sales of almost $60 billion dollars/per year (supra). Monoclonal antibodies are typically expressed in eukaryotic cells cultured from multicellular organisms (humans, hamsters, or insects) in order to achieve correct protein folding and proper attachment of carbohydrates, which lend to variable production quality that is slow and scales poorly. In contrast, the invention disclosed herein can be expressed in prokaryotic cells or yeast, reducing the cost of GMP (Good Manufacturing Practice) by up to ten-fold. The invention disclosed represent a substantial economic improvement over current monoclonal antibody synthesis, particularly for chronic drug administration, such as a viral prophylactic or treatment of a chronic disease, including, but not limited to auto-immune diseases and cancer.

While previous patents have utilized human trimeric constructs for multimeric binding with monoclonal antibodies¹⁻⁵, and heteromultimers⁶, none have used them as constructs for purely synthetic human derived antibody substitutes as described in the invention herein.

### BRIEF SUMMARY OF THE INVENTION

As illustrated in FIG. 1, the present invention of a multimeric protein complex as antibody substitute is directed to a modified symmetric multimeric protein complex (αₘ) of human origin with m-fold point group symmetry such as Cₘ or Dₘ with each monomeric protein a fused at its N-terminus or C-terminus with the C-terminus or N-terminus of a repeated modified beta solenoid sequence (mBSP) denoted as βₙ of human origin, with a length of n fused repeats (with 0≤n)(e.g., n can be 0, 1, 2, 3, 4, or more) and β corresponding to the mBSP. This βₙ sequence is fused at the other N-terminus or C-terminus with the C-terminus or N-terminus of a pathogen binding domain (PBD) denoted as γ in the sequence schematic of FIG. 1, that can have p copies (γₚ with 1≤p)(e.g., p can be 1, 2, 3, 4, or more). Hence, per FIG. 1, the generic schematic protein structure is (α-βₙ-γₚ)ₘ with α-βₙ-γₚ the structure of the monomeric protein unit of the m-fold symmetric multimeric protein complex if the N-terminus of the symmetric human multimeric protein complex is the overall starting point of the fused protein, or the structure is (γₚ-βₙ-α)ₘ if the N-terminus of the PBD is the overall starting point of the fused protein. The “modular” structure indicates the multimeric protein complex is composed of a monomeric protein of the original symmetric human multimer, a fused n-meric protein domain of the human mBSP protein, and a PBD.

The PBD is rationally engineered from a known human receptor protein. We can choose all these proteins to be expressible in E. coli or other prokaryotic organisms, as well as in single cell eukaryotic organisms such as P. pastoris. In some embodiments, the αₘ is the trimerization (m=3) domain of collagen⁷ (PDB code 3N3F), or other multimeric human protein complexes including trimeric EDA-1^{8, 9} (PDB code 1RJ7, m=3), trimeric Langerin^{10, 11} (3KQG, m=3), and tetrameric diubiquitin^{12, 13} (2XEW, m=4); the mBSP may be obtained from structures including the p27 unit of the human dynactin complex¹⁴ (3TV0), or from the

Retinitis Pigmentosa 2 Protein^{15, 16} (RP2) (2BX6); the PBD may be obtained from the N-terminus of the human ACE2 receptor protein^{17, 18} for neutralization of SARS-CoV-1 and SARS-CoV-2 for example by binding to the corresponding coronavirus Spike protein Receptor Binding Domain (RBD), or from parts of any other known human receptor protein, such as the DPP4 human protein, which binds to the MERS Spike RBD.

The multimeric protein complex as antibody substitute described herein allows a variable size dependent upon the number, n, of mBSPs, so that one multimeric viral envelope protein (VEP) with m-fold symmetry denoted as (VEP)ₘ can be neutralized or multiple (VEP)ₘ can be neutralized. This is illustrated for m=3 for the SARS-CoV-2 Spike protein in FIGS. 2 and 6 (one SARS-CoV-2 spike neutralized), and 7 (three SARS-CoV-2 spikes neutralized).

In some embodiments, the overall sequence α-βₙ-γₚ of one of the monomeric protein domains of the multimeric protein complex as antibody substitute comprises a sequence shown in SEQ ID NO:1 or at least 90% or 95% or 98% or 99% identical to SEQ ID NO :1, for which n=0, p=1, α is a monomeric protein of the collagen trimerization domain (3N3F on the PDB), and the PBD (γ) is at least 90% or 95% or 98% identical to the N-terminus domain of residues 19-85 of the human ACE2 receptor protein with modifications S19G, T20C, P84A, L85C. These changes provide a disulfide bridge to stabilize the ends of the protein.

In some embodiments, the overall sequence of α-βₙ-γₚ of one of the monomeric protein domains of the multimeric protein complex as antibody substitute comprises a sequence shown in SEQ ID NO: 2 or at least 90% or 95% or 98% or 99% identical to SEQ ID NO: 2 for which n=0, p=1, a is a monomeric protein of the human collagen trimerization domain (3N3F on the PDB), and the PBD (γ) is at least 90% or 95% or 98% identical to the N-terminus domain of residues 19-91 of the human ACE2 receptor protein with modifications N52Q, M62K, M69K. These changes remove a potential N-linked glycosylation site and mitigate the tendency of the PBD to dimerize via hydrophobic interaction since the lysines are positively charged.

In some embodiments, the overall sequence α-βₙ-γₚ of one of the monomeric protein units of the multimeric protein complex as antibody substitute comprises a sequence shown in SEQ ID NO:3 or at least 90% or 95% or 98% or 99% identical to SEQ ID NO:3 for which n=0, p=1, a is a monomeric protein of the human collagen trimerization domain (3N3F on the PDB), and the PBD (γ) is at least 90% or 95% or 98% identical to the N-terminus domain of residues 19-91 of the human ACE2 receptor protein with modifications N52Q, M62R, M69R. These changes remove a potential N-linked glycosylation site and mitigate the tendency of the PBD to dimerize via hydrophobic interaction since the arginines are positively charged.

In some embodiments, the overall sequence α-βₙ-γₚ of one of the monomeric protein units of the multimeric protein complex as antibody substitute comprises a sequence shown in SEQ ID NO:4 or at least 90% or 95% or 98% or 99% identical to SEQ ID NO:4

In some embodiments, the overall sequence α-βₙ-γₚ of one of the monomeric protein units of the multimeric protein complex as antibody substitute comprises a sequence shown in SEQ ID NO 5 or at least 90% or 95% or 98% or 99% identical to SEQ ID NO:5.

In some embodiments, the present invention may consist of a single PBD rationally engineered from a known pathogen-binding human receptor protein, or of p copies (γ)ₚ of a single PBD=γ rationally engineered from a known pathogen binding human receptor protein that binds at a common interface.

In some embodiments, the γₚ may be rationally engineered from the human ACE2 receptor protein, and each element of the γₚ comprises the sequence shown in SEQ ID NO: 4 or at least 90% or 95% or 98% or 99% identical to SEQ ID NO: 6 or SEQ ID NO: 7.

In some embodiments, the PBD is modified to reduce probability of N-linked glycan attachment, for example by mutation of N52 in the wildtype N-terminus of the human ACE2 protein to N52Q so that the NIT glycan attachment sequence is disrupted.

In some embodiments, the PBD is modified to have a different sequence relative to wild type human form to deter multimerization of the monomeric proteins in γₚ.

In some embodiments, the α or β=mBSP domains of the (α-βₙ-γₚ)ₘ or (γₚ-βₙ-α)ₘ multimeric protein complex as antibody substitute may be modified to include at least one amino acid that promotes attachment to a solid support, a nanoparticle, or a biological molecule.

In some embodiments, the α or β=mBSP domains of the (α-βₙ-γₚ)ₘ or (γₚ-βₙ-α)ₘ multimeric protein complex as antibody substitute may be modified to include at least one amino acid that promotes attachment to human albumin.

In some embodiments, the pathogen is a virus. In some embodiments the virus is SARS-CoV-2 or SARS-CoV-1 or MERS or HBV or HCV or HIV or Ebola or Marburg or CMV.

In some aspects, the disclosure provides a multimeric protein complex as antibody substitute complex comprising a plurality (e.g., m≥3) of monomeric proteins with modular protein domains of the form (α-βₙ-γₚ)ₘ, or (γₚ-γₙ-α)ₘ wherein the monomeric proteins α-βₙ-γₚ or γₚ-γₙ-α comprise fused protein domains with a being a monomeric protein from a symmetric human multimeric protein complex of point group symmetry Cₘ or Dₘ, βₙ being a fused domain of n modified beta solenoid proteins (mBSPs) with 0≤n, and γₚ being a complex of p≥1 pathogen binding domains (PBDs) either fused or bound by intermolecular forces.

In some aspects, the disclosure provides a multimeric protein complex as antibody substitute complex comprising a plurality (e.g., m≥2) of monomeric proteins with modular protein domains of the form (α-βₙ-γₚ)ₘ or (γₚ-βₙ-α)ₘ wherein the monomeric proteins α-βₙ-γₚ or γₚ-βₙ-α comprise fused protein domains wherein:
α is a monomeric protein from a symmetric human multimeric protein complex of point group symmetry Cₘ or Dₘ,
βₙ is a fused domain of n modified beta solenoid proteins (mBSPs) with n≥0, and
   γₚ is a complex of p pathogen binding domains (PBDs) either fused or bound to each other by intermolecular forces and wherein p≥1.

In some embodiments, wherein the multimeric protein complex as antibody substitute is symmetrical. In some embodiments, the multimeric protein complex as antibody substitute has two-fold symmetry. In some embodiments, the multimeric protein complex as antibody substitute has three-fold symmetry. In some embodiments, the multimeric protein complex as antibody substitute has four-fold symmetry. In some embodiments, the multimeric protein complex as antibody substitute has five-fold symmetry. In some embodiments, the multimeric protein complex as antibody substitute has six-fold symmetry. In some embodiments, the multimeric protein complex as antibody substitute has twelve-fold symmetry.

In some embodiments, the modular protein domain a is a monomeric protein from a wild type symmetric multimeric protein complex αₘ. In some embodiments, the modular domains are subsequences of human proteins.

In some embodiments, α is a monomeric protein from the m=3 human collagen trimerization domain which is at least 90%, 95%, 98%, or 99% identical to SEQ ID NO: 9. In some embodiments, a is a monomeric protein from the m=3 human growth factor EDA-A1 which is at least 90%, 95%, 98%, or 99% identical to SEQ ID NO: 10. In some embodiments, α is a monomeric protein from the m=3 human Langerin which is at least 90%, 95%, 98%, or 99% identical to SEQ ID NO: 11. In some embodiments, a is a monomeric protein from the m=4 human tetrameric diubiquitin which is at least 90%, 95%, 98%, or 99% identical to SEQ ID NO: 12.

In some embodiments, n=0, p=1 and the protein binding domain (PBD) γ is at least 90%, 95%, 98% or 99% identical to the N-terminus domain (residues 19-85 or residues 19-91) of the human ACE2 receptor protein of SEQ ID NO: 6 or SEQ ID NO: 7.

In some embodiments, the monomeric sequence is at least 90%, 95%, 98% or 99% identical to SEQ ID NO:1. In some embodiments, the monomeric sequence is at least 90%, 95%, 98% or 99% identical to SEQ ID NO:2. In some embodiments, the monomeric sequence is at least 90%, 95%, 98% or 99% identical to SEQ ID NO:3.

In some embodiments, a is a monomeric protein from other m-fold symmetric protein multimeric protein complexes such as trimeric EDA1 of SEQ ID NO: 10 or Langerin of SEQ ID NO: 11 or tetrameric diubiquitin of SEQ ID NO: 12.

In some embodiments, modified human beta solenoid (mBSP) is at least 80%, 90%, 95%, 98%, or 99% identical to the dynactin p27 domain (3VT0) of SEQ ID NO: 8.

In some embodiments, the monomeric sequence α-βₙ-γₚ is at least 90%, 95%, 98% or 99% identical to SEQ ID NO: 4 and n=1 and p=1.

In some embodiments, the monomeric sequence γₚ-βₙ-α is at least 90%, 95%, 98% or 99% identical to SEQ ID NO: 5, and n=4 and p=1.

In some embodiments, the sequence is of the form γ₂ and γ is at least 90%, 95%, 98% or 99% identical to SEQ ID NO: 6 or SEQ ID NO: 7.

In some embodiments, the modified human beta solenoid is modified to be at least 80%, 90%, 95%, 98% or 99% identical to the human Retinitis Pigmentosa Protein 2 (RP2) (2BX6).

In some embodiments, the pathogen binding domain is at least 90%, 95%, 98% or 99% identical to the helix-turn-helix (HTH) complex from the N-terminus of the ACE2 receptor protein of SEQ ID NO: 6 or SEQ ID NO: 7.

In some embodiments, at least one (e.g., 1, 2, 3, 4, 5, or more) amino acid of the a or modified human beta solenoid domain is modified to allow attachment to a nanoparticle, a solid support, or other biological molecule.

In some embodiments, the multimeric protein complex is attached to a nanoparticle, a solid support, or other biological molecule.

In some embodiments, the multimeric protein complex is attached to human serum albumin.

In some embodiments, at least one (e.g., 1, 2, 3, 4, or more) amino acid of one or more of the module domains is modified to allow attachment to a nanoparticle, a solid support, or other biological molecule.

Also provided is a multimeric protein complex as antibody substitute comprising a plurality of pathogen binding domains (e.g., 2, 3, 4, or more). In some embodiments, the pathogen binding domain is modified to be at least 90%, 95%, 98% or 99% identical to the HTH domain (residues 19-85 or 19-91) of the N-terminus of the ACE2 receptor protein of SEQ ID NO: 6 or SEQ ID NO: 7. In some embodiments, at least one (e.g., 1, 2, 3, 4, or more) amino acid of either or all pathogen binding domains are modified to allow attachment to a nanoparticle, a solid support, or other biological molecule.

Also provided is a method for neutralizing a pathogen comprising contacting said pathogen with the multimeric protein complex. In some embodiments, the antibody substitute is as described above or elsewhere herein, e.g., wherein one or more pathogen binding domains binds to one or more sites on the pathogen.

Also provided is a method for immobilizing a pathogen comprising contacting said pathogen with the multimeric protein complex. In some embodiments, the antibody substitute is as described above or elsewhere herein, e.g., wherein one or more pathogen binding domains binds to one or more sites on the pathogen. In some embodiments, the pathogen is a virus.

### DEFINITIONS

α: “monomeric protein unit of a symmetric multimeric protein complex.” This is a single protein unit of a symmetric multimeric protein complex.

αₘ: “symmetric multimeric protein complex”. A set of m (m can be 2, 3, 4, 5, etc.) identical proteins that form a complex invariant under m-fold rotations about the symmetry axis and are held together by non-covalent bonds. Examples am is the trimerization (m=3) domain of collagen⁷ (PDB code 3N3F), or other multimeric human protein complexes including trimeric EDA-1^{8, 9} (PDB code 1RJ7, m=3), trimeric Langerie^{10, 11} (3KQG, m=3), and tetrameric diubiquitin^{12, 13} (2XEW, m=4). The full sequence of each of these proteins is available from the Protein Database.

BSP: “beta-solenoid protein”. Proteins having backbones that turn helically in either a left- or right-handed sense around the long axis of the protein structure from the N-terminus to the C-terminus to form contiguous β-sheets, typically with 1.5-2 nm sides. Examples of non-amyloidogenic WT-BSPs that can form amyloid fibrils upon modification include: one-sided antifreeze proteins (AFPs) (Tenebrio molitor AFP-Protein Database (PDB) Accession No. 1EZG¹⁹⁻²²), two-sided AFPs (Snow Flea AFP-PDB 2PNE and 3BOI^{23, 24}), rye grass AFP (PDB-3ULT^{25, 26}), three-sided “type II” left handed beta-helical solenoid AFPs, for example from the spruce budworm (PDB 1M8N²¹), three-sided bacterial enzymes (PDB 1LXA²⁷, 1FWY²⁸, 1G95²⁹, 1HV9³⁰, 1J2Z³¹, 1T3D³², 1THJ³³, 1KGQ³⁴, 1MR7³⁵, 1SSM³⁶, 2WLC³⁷, 3R3R³⁸, 1KRV³⁹, 3EH0⁴⁰, 3Q1X⁴¹, 3BXY⁴², 3HJJ⁴³, 30GZ⁴⁴, 4M98⁴⁵, 4IHH⁴⁶ (acyltransferases, γ-class carbonic anhydrases and homologs), three-sided human motor protein subunits (e.g., PDB 3TV0¹⁴), a three-sided “type I” left handed beta-helical enzyme ydcK from Salmonellae cholera (2PIG^{47, 48}), four sided proteins (PDB 2BM6⁴⁹, 2W7Z⁴⁹, 2J8I⁴⁹), four-sided pentapeptide repeat proteins (2G0Y⁵⁰ and 3DU1⁵¹), and 1XAT⁵². The full sequence of each of these proteins is available from the Protein Database.

mBSP: “modified β solenoid protein” (also referred to as “mBSP monomeric protein”). Genetically engineered β solenoid proteins that allow for insertion into a (α-βₙ-γₚ) fused monomeric protein with β=mBSP . The mBSP is modified from an existing BSP such as 3TV0. An mBSP monomeric protein can be engineered to be of any length, typically from three rungs of a beta solenoid structure up to 24 or more depending upon the length needed for a particular binding application.

βₙ: “modified β solenoid protein n-meric protein domain”. An mBSP monomeric protein domain consisting of n (n can be 0, 1, 2, 3, 4 etc.) identical fused and epitaxially bonded copies of a single mBSP derived from a wild type protein such as 3TV0.

mBSP epitaxy: “mBSP epitaxy”. The structural alignment of multiple engineered monomeric mBSPs (denoted as β in the protein schematic of this proposal per [0005] and FIG. 1) to form a single, regular, contiguous repeated fused structure. For example, to assure epitaxy (see below) of the layers of the repeated fused βₙ of the 3TV0 dynactin p27 protein, residues 146 to 159 must be removed. This section reverses the left handed helicity of the protein and would otherwise prevent the formation of a regular, contiguous repeated fused structure. It can be retained on the final C-terminus of the fused βₙ domain to inhibit unwanted multimerization of the arms of the (α-βₙ-γₚ)ₘ or (γₚ-βₙ-α)ₘ multimeric protein complex as antibody substitute to one another. The aggregation-inhibiting cap sequence in this case is ADDCLRRVQTERPQP (SEQ ID NO:13). The three dimensional structure of any given BSP can be used to design an mBSP of that desired shape. Means for modeling engineered proteins and characterizing their final properties are well known to those skilled in the art. Exemplary techniques for these procedures are described in, e.g., U.S. Pat. No. 10,287,332⁵³.

Functionalized mBSP: “functionalized mBSPs”. BSPs that are designed to specifically carry designated functional units, for example pathogen binding proteins, which are fused to either the amino- or carboxyl-terminus (or both) of mBSPs. In some embodiments, the mBSP monomeric proteins can further include one or more amino acid residues at the end.

PBD: “pathogen binding domain”. Proteins that are rationally engineered by extraction from full length human receptors that have binding to known viral envelope proteins, For example, in the examples of the present invention in SEQ ID NOS: 1-7, the PBD is taken from the N-terminus of the ACE2 receptor protein, with a few possible mutations, that binds to the RBD of the Spike VEP from SARS-CoV-2.

γₚ: “Pathogen binding domain with p copies”. A pathogen binding domain is denoted by γ in the multimeric protein complex schematic language of this application, that can form multimeric protein complexes with p-copies (p can be 1,2,3,4 etc.). For example, in the present invention, in sequence 6, the HTH₂ complex binds together because of a significant content of hydrophobic residues on the HTH face opposite to the binding site for the RBD protein of the SARS-CoV-2 spike complex.

Multimeric protein as antibody substitute. A multimeric protein complex of the form (α-βₙ-γₚ)ₘ with α-βₙ-γₚ the fused monomeric protein unit of the m-fold symmetric protein if the N-terminus of the symmetric human multimeric protein complex is the overall starting point of the fused protein, and where α is a monomeric protein from a symmetric human multimeric protein complex such as the human collagen trimerization domain (3N3F PDB ID) used in SEQ ID NOS: 1-3, β is an mBSP such as the modified p27 dynactin domain (PDB ID 3VT0) used in SEQ ID NOS: 4 and 5, and γ is a PBD extracted from a human receptor protein such as residues 19-91 of the ACE2 receptor protein used in SEQ ID NOS: 1-7. If the N-terminus of the monomeric unit of the protein is on the PBD, the multimeric protein complex as antibody substitute has the form (γₚ-βₙ-α)ₘ. The index n refers to the fusion of n copies of an mBSP into a single domain, and the index p refers to p copies of a PBD either fused or bonded together by intermolecular interactions.

Fused domains—two otherwise independent protein domains are said to be fused if they are linked by a peptide bond.

VEPₘ: “m-fold viral envelope protein”. A symmetric complex on the surface of a virus that binds to a surface receptor protein on a human cell. The VEPₘ has m-fold point group symmetry such as Cₘ or Dₘ.

Neutralization: “Neutralization”. The coating of the VEP binding sites of the virus sufficiently to block attachment to a cell surface protein and thus block infection.

Sequence Identity: “identical or percent identity”. In the context of two or more nucleic acids or polypeptide sequences (e.g., two mBSPs and polynucleotides that encode them), this refers to two or more sequences or subsequences that are the same or have a specified percentage of amino acid residues or nucleotides that are the same, when compared and aligned for maximum correspondence, as measured using one of the sequence comparison algorithms listed below, or by visual inspection.

Substantially Identical: “substantially identical”. In the context of two nucleic acids or polypeptides of the invention, this refers to two or more sequences or subsequences that have at least 60%, 65%, 70%, 75%, 80%, or 90-95% nucleotide or amino acid residue identity (e.g., to any of the sequences here, including but not limited to SEQ ID NO:1, 2, 3, 4, and 5), when compared and aligned for maximum correspondence, as measured using one of the sequence comparison algorithms listed below, or by visual inspection. Preferably, the substantial identity exists over a region of the sequences that is at least 50 residues in length, more preferably over a region of at least 100 residues, and most preferably sequences that are substantially identical over at least 150 residues. In a most preferred embodiment, the sequences are substantially identical over the entire length of the coding regions.

Sequence Comparison: “sequence comparison”. Typically, one sequence acts as a reference sequence, to which test sequences are compared. When using a sequence comparison algorithm, test and reference sequences are input into a computer, subsequence coordinates are designated, if necessary, and sequence alignment program parameters are specified. The sequence alignment algorithm then calculates the percent sequence identity for the test sequence(s) relative to the reference sequence, based on the specified program parameters.

Optimal Alignment: “Optimal alignment”. This means the most likely alignment of protein sequences for comparison. This can be conducted, e.g., by the local homology algorithm of Smith & Waterman⁵⁴, by the homology alignment algorithm of Needleman & Wunsch⁵⁵, by the search for similarity method of Pearson & Lipman⁵⁶, and by computerized implementations of these algorithms (GAP, BESTFIT, FASTA, and TFASTA in the Wisconsin Genetics Software Package, Genetics Computer Group, 575 Science Dr., Madison, WI), or by visual inspection (see generally, Current Protocols in Molecular Biology, F. M. Ausubel et al., eds., Current Protocols, a joint venture between Greene Publishing Associates, Inc. and John Wiley & Sons, Inc., (1995 Supplement) (Ausubel)).

Alignment Algorithms: “Alignment algorithms”. These are programs that are suitable for determining percent sequence identity and sequence similarity, for example the BLAST and BLAST 2.0 algorithms, which are described in Altschul et al.^{57, 58}. Software for performing BLAST analyses is publicly available through the National Center for Biotechnology Information (http://www.ncbi.nlm.nih.gov/). These algorithms involve first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold^{57, 58}. These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always>0) and n (penalty score for mismatching residues; always<0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) of 10, M=5, N=-4, and a comparison of both strands. For amino acid sequences, the

BLASTP program uses as defaults a word length (W) of 3, an expectation (E) of 10, and the BLOSUM62 scoring matrix⁵⁹

Statistical Analysis: “Statistical analysis”. This refers to quantitative statistical analysis of the similarity between two sequences to quantify the degree of similarity apart from the visual alignment and percentage overlap of the protein sequences^{60, 61}. One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

Avidity. Avidity refers to the enhanced binding of a multimeric protein complex relative to a monomeric protein when the multimeric protein complex consists of m copies of the monomer. Because the binding free energies of a monomeric protein add linearly absent cooperative effects between the multimeric units, the binding strength as characterized by the binding affinity measured by the equilibrium association constant K_{Am}=(K_{Dm})⁻¹˜(K_{A1})^{m}, so that the binding strength is dramatically enhanced by avidity.

RBD: “Receptor Binding Domain”. Here, this is applied to an individual protein of the spike trimer complex for a coronavirus such as SARS-CoV-2 that ‘flips’ between a concealed conformation and an exposed (binding) conformation from the spike complex. It is this RBD that binds to the HTH pathogen binding domain of the ACE2 protein receptor on epithelial cells.

Point group symmetry For the symmetric human multimeric protein complexes and the multimeric protein complexes as antibody substitutes, each protein takes the form Φₘ where Φ is a single (monomeric) protein and the m-copies bind at the interface such that when structural fluctuations are removed there is an m-fold symmetry about an axis through the center of the assembly: rotations of 2πq/m where 0≤q<m take the structure into itself. Such a pure rotation symmetry about one axis is denoted Cₘ. If there are in addition reflection symmetries in a plane perpendicular to the m-fold axis, the complex can have the symmetry Dₘ.

### BRIEF DESCRIPTION OF THE DRAWINGS

FIG. 1. Schematic drawing of a multimeric protein complex as antibody substitute .

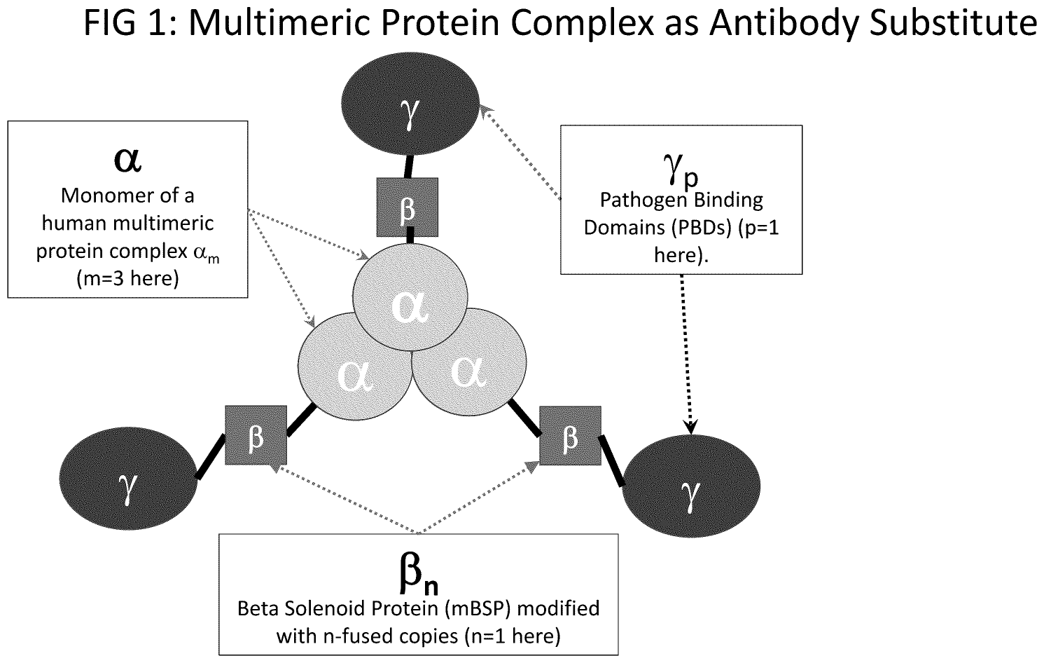

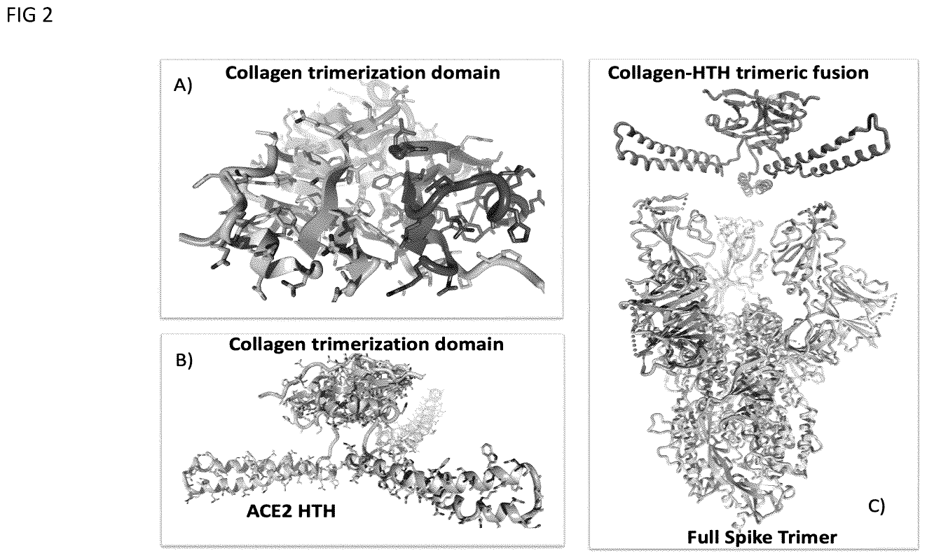

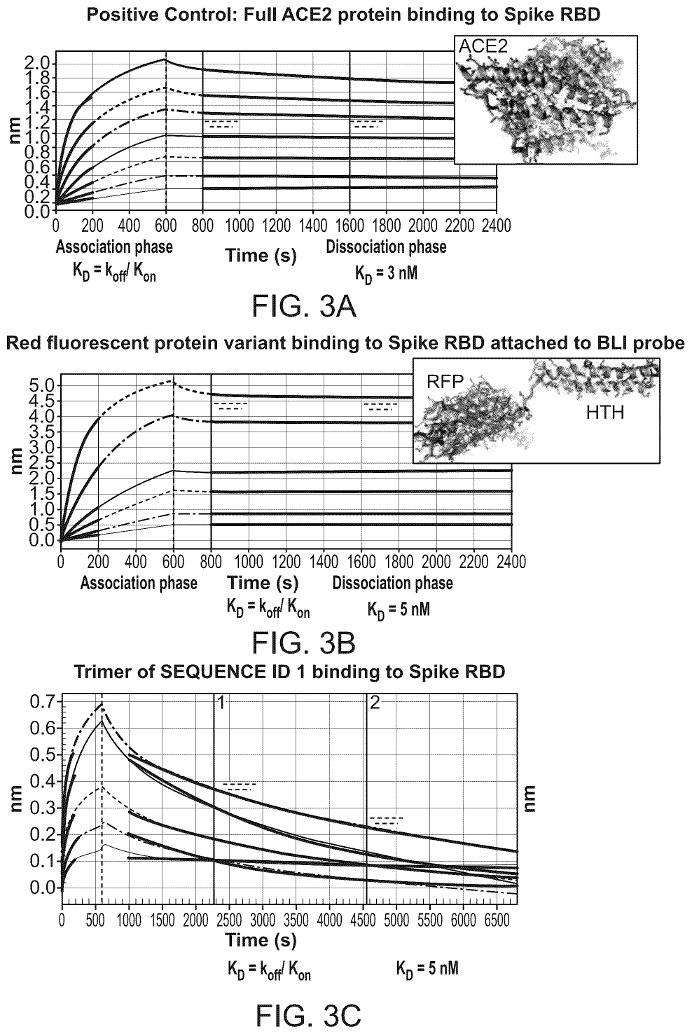

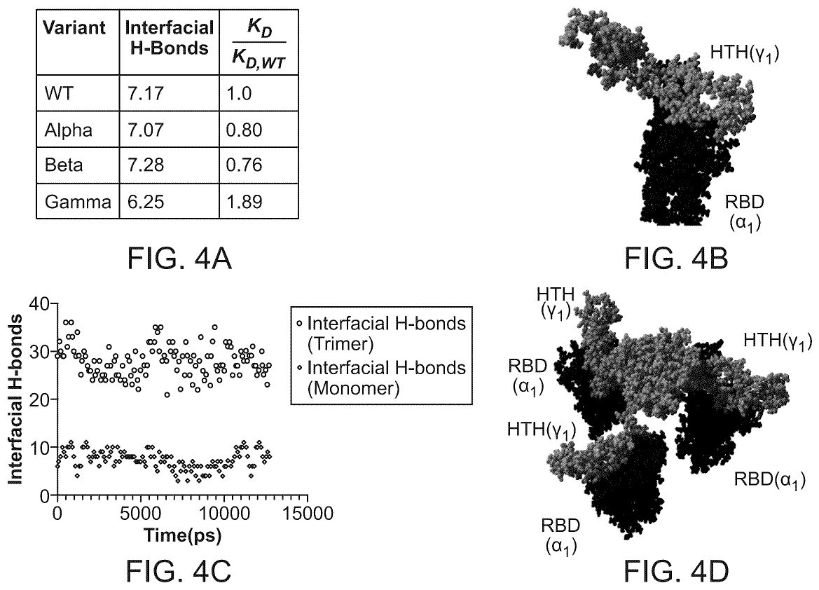

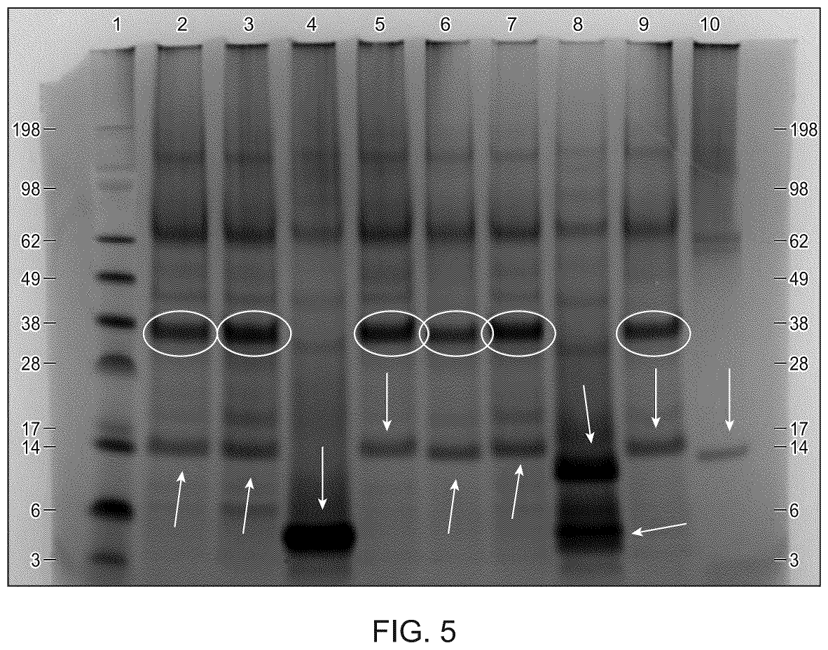

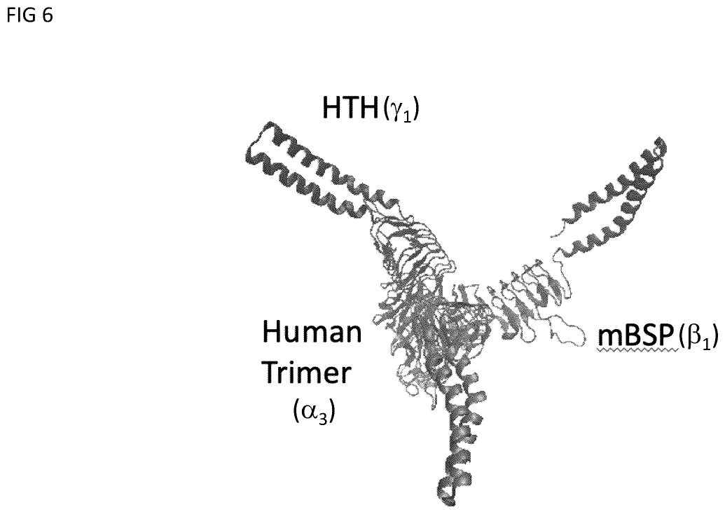

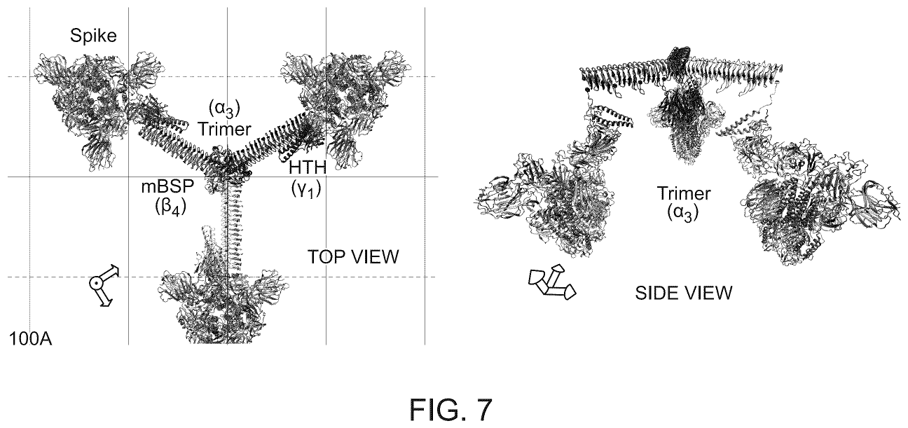

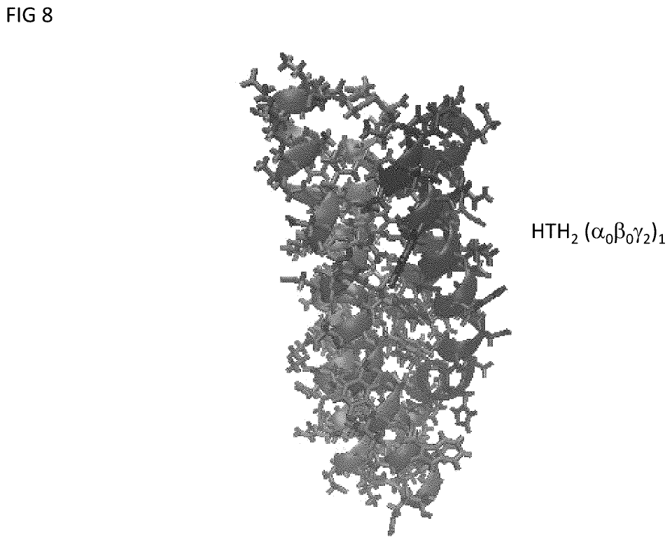

In this case the multimeric protein complex is a trimer (m=3) comprised of a monomeric protein from a symmetry human trimer such as the human collagen trimerization domain (PDB ID 3N3F), fused to a modified beta solenoid (mBSP) domain in which n copies of a single mBSP such as the p27 domain from dynactin (PDB ID 3VT0) are fused together, and p copies of a pathogen binding domain (PBD) such as residues 19-91 of the human ACE2 receptor protein. If the sequence begins with the α-monomer, it will have the form (α-βₙ-γₚ)ₘ with α-βₙ-γₚ the fused (by peptide bond) monomeric protein of the α, βₙ, and γₚ domains. If the sequence begins with the PBD, it will have the form (γₚ-βₙ-α)ₘ. The multimeric value m is chosen to match the symmetry of the corresponding viral envelope protein (VEP)ₘ where, e.g., VEP can be a monomeric protein of the Spike trimer from the SARS-CoV-2 virus.

FIG. 2A-C Sample trimer neutralizing structure (γ-α)₃ where α is a monomeric protein from the A) human collagen trimerization domain (PDB Record 3N3F) and γ comes from N-terminus residues (19-85) of the human ACE2 protein with mutations per SEQ ID NO: 1. B) Full Trimer. C) This is designed to neutralize a single spike trimer complex of SARS-CoV-2 virions.

FIG. 3A-C. A) Positive Control of binding of ACE2 protein to SARS-CoV-2 RBD protein measured in a biolayer interferometry (BLI) experiment, with an inferred 3 nM equilibrium dissociation constant, K_{D}. B) Binding of HTH fused with red fluorescent protein (RFP) in a BLI experiment with inferred dissociation constant K_{D}=5 nM. C) Binding of trimer synthesized from SEQ ID NO: 1 to monomeric RBD in a BLI experiment. The inferred dissociation constant K_{D}=5 nM showing that the monomeric binding is comparable to the ACE2.

FIG. 4A-D. Results from Simulations of (γ-α)₃ where α=monomeric protein from the human collagen trimerization domain, γ=PBD from residues (19-91) from human ACE2 protein with mutations per SEQ ID NO: 3. A) 10 nanosecond YASARA molecular dynamics simulations together show interfacial hydrogen bond counts for the ACE2 derived PBD binding to SARS-CoV-2 Spike RBD variants (wild type, alpha, beta, gamma) show little variation. As a corollary a combined analysis from HawkDock MM/GBSA correlated with Deep Mutational Scan measurements of binding energy changes from point mutations on RBD allows predictions of variant dissociation constant with wildtype, and the changes are small. B) Structure of SEQ ID NO: 3 simulated binding to single Spike RBD. C) Interfacial hydrogen bond counts from 12 nanosecond YASARA simulations of monomeric RBD bound to single HTH, and trimeric form of SEQ ID NO: 3 bound to three RBDS held by constraining potential in positions found experimentally for all 3 spike RBDs out. Avidity is demonstrated by >3X enhancement of interfacial hydrogen bound count for SEQ ID NO: 3, which correlates well with binding energy. D) Simulated structure of trimeric RBDs constrained to positions of full spike trimer.

FIG. 5 SDS PAGE confirming trimer expression from SEQUENCE ID 2 and SEQUENCE ID 3 from P. pastoris. Lane 1—mass standard for SDS-PAGE, protein mass markers (approximate) in kilodaltons (KDa). Lane 10—Cytochrome C Oxidase standard. Monomers at ˜KDa. Lane 2, 3: expression from one P. pastoris culture of the protein of SEQUENCE ID 2 in nonreducing (2) and reducing (3) conditions. Same for different P. pastoris culture in Lanes 6,7. Note monomeric protein bands near 14 KDa (arrows) and identified (red ovals) trimer bands near 38 KDa. Lanes 5,9: Expression from P. pastoris culture of SEQUENCE ID 3 in nonreducing (5) and reducing (9) conditions. Again, note monomeric protein bands near 14 KDa and trimer bands near 38 KDa. The lighter apparent mass of the trimer per unit is attributable to excess negative charge. Lanes 4,8: Failed expression of modified version of SEQUENCE ID 1.

FIG. 6. Sample trimeric neutralizing structure (α-β₁-γ)₃ where a is a monomeric protein of the trimerization domain (PDB Record 1RJ7), β is an mBSP from the dynactin p27 BSP (PDB record 3VT0), and γ is the PBD taken from the N-terminus residues (19-83) of the human ACE2 protein. This is designed to neutralize a single spike trimer complex of SARS-CoV-2 virions.

FIG. 7. Sample trimeric neutralizing structure (γ-β₄-α)₃ where α is a monomeric protein of the trimerization domain (1RJ7), β is an mBSP from the dynactin p27 BSP (3VT0) with four copies fused together in a single unit, and γ=HTH comes from N-terminus residues (19-83) of the human ACE2 protein. This structure is designed to neutralize up to three spike 25 complexes of SARS-CoV-2 virions.

FIG. 8. Sample model γ₂ dimer constructed from γ=HTH from residues (19-83) of the ACE2 human receptor protein . The two HTH complexes (one red, one blue) bind together because of hydrophobic interactions on the inner surface of the peptides.

### DETAILED DESCRIPTION OF THE INVENTION

The inventors have discovered that engineered protein trimers and dimers composed of linked modular sequences from human proteins can neutralize viral pathogens in a prophylactic or therapeutic context to form a multimeric protein complex as antibody substitute. By fusing a purely human multimeric protein complex comprised of m monomeric proteins αₘ to mBSPs and PBDs to match the m-fold symmetry and geometry of the viral envelope protein, it is possible to neutralize the multimeric VEP protein with a multimeric protein as an antibody substitute properly attuned to the VEP symmetry. In contrast monoclonal antibodies which at best exhibit dimeric (m=2) protein binding, but without a geometry tuned to the VEPₘ geometry they typically exhibit monomeric protein binding. The multimeric protein binding increases the net binding affinity to the VEPₘ complex. The inventors have previously demonstrated, for example, that a similar binding domain to that of the PBD from the ACE2 of SEQ ID NOS:1-6 attached to mBSP polymers can successfully bind vascular endothelial growth factor (VEGF) protein. The symmetric human multimeric protein complexes expressible in E. coli can be, for example, the human collagen trimerization domain (PDB code 3N3F) or other multimeric human protein complexes including but not limited to trimeric EDA-1 (PDB code 1RJ7), trimeric Langerin (3KQG), and tetrameric diubiquitin (2XEW), as detailed in SEQ ID NO: 9, 10, 11, and 12. FIG. 2 shows the design from SEQ ID NO: 1 using the human collagen trimerization domain and a single modified HTH per collagen monomer.

The inventors have discovered that in the case of the PBD in the embodiment of the N-terminus sequence (see, e.g., SEQ ID NO: 6 and SEQ ID NO: 7) from the ACE2 protein that the binding is effective and essentially unchanged from the ACE2 dimer itself (FIG. 3). We denote this PBD as the HTH complex since it has an alpha helix (H)-turn(T)-alpha helix (H) structure. This indicates that the construct itself is sufficient for neutralizing a single RBD. Note that the trimer synthesized in E. coli from SEQ. ID NO:1 binds to the monomeric RBD with the same affinity as the RFP-HTH construct. Previous computer simulations suggest that the HTH binds RBD.⁶²

The inventors have discovered through extensive simulations using the YASARA molecular dynamics program⁶³ that mutations associated with extensive variants of the SARS-CoV-2 virus do not significantly alter the binding of the PBD from residues 19-91 of the ACE2 to the RBD of the SARS-CoV-2 spike protein (FIG. 4a) and that the multimeric protein complex as antibody substitute trimer construct of SEQ ID NO 3 binds to the full spike protein with all RBDs out with >3X the interfacial hydrogen bond count than the monomer, which will lead to up to a million fold greater binding affinity to the full spike protein with all RBDs out (FIG. 4c) confirming the avidity of the trimer per the definition.

The inventors have discovered that the multimeric protein complex as antibody substitute designs of SEQ ID NO: 2 and SEQ ID NO: 3 are expressible in P. pastoris with the trimers secreted from the cells and needing no post-translation modification, confirming the advantage of the invention over monoclonal antibodies. (FIG. 5). Note that a modification of SEQ ID NO: 1 does not express well from the P. Pastoris.

The inventors have discovered that by inserting a human βₙ=mBSPₙ domain consisting of n fused copies of a single mBSP domain between a monomeric proteins and the γ=PBD element of the multimeric protein complex as antibody substitute that they can adapt the size of the multimeric protein complex as antibody substitute to match the size of the VEPm to bind to more than one VEP element at once (FIG. 2 for the SARS-CoV-2 virus, with n=0, FIG. 6 with n=1), or to have sufficient length to bind to more than one VEPm complex at a time (FIG. 7 for the SARS-CoV-2 virus, with n=4). The human mBSP expressible in E. coli, for example, is shown in SEQ ID NO: 8 modified from the dynactin p27 protein (PDB ID 3TV0) or can be obtained from modifying the Retinitis Pigmentosa 2 protein (RP2)(PDB ID 2BX6).

The inventors have discovered that in the case of the N-terminus ACE2 HTH example of SEQ ID NO: 6 or SEQ ID NO: 7 for a PBD, that there is a tendency to dimerize when detached from the ACE2 protein (FIG. 8). The source of this dimerization is a relatively large patch of hydrophobic residues which tend to associate together. Given that this dimer is a small construct (mass of approximately 15 KDa) this discovery allows the dimer itself to be a potent, easily dispersed neutralizer of spike proteins via binding to RBD.

By making use of purely or nearly (see the following paragraph) human proteins in each of the modular domains of (α-βₙ-γₚ) or (γₚ-βₙ-α) for the multimeric protein complex as antibody substitute, the invention avoids immunogenic response of the human host in prophylactic or therapeutic applications.

Immunogenic tolerance of the host to these multimeric protein complexes as antibody substitutes can be maintained by modifying up to 5 residues of the PBD to either (a) increase the innate binding strength to the VEP complex, or (b) to inhibit the dimerization of a PBD such as the HTH PBD construct from the ACE2 protein. For example, the substitutions of lysines or arginines at positions 62,69 of the HTH PBD in SEQ ID NO: 2 or 3 helps to significantly diminish the hydrophobic dimerization tendency.

The probability for attachment of N-linked glycans to the multimeric proteins as antibody substitutes can be substantially reduced by modifying NIT sequences to QIT, as for example in SEQ ID NO:2 or 3.

Exemplary multimeric protein complex as antibody substitute sequences of the form (α-βₙ-γₚ)ₘ or (γₚ-βₙ-α)ₘ include but are not limited to polypeptides comprising an amino acid sequence at least 90%, 95%, 98%, 99% or 100% identical any one of SEQ ID NOS: 1, 2, 3, 4, 5, 6, or 7 as provided below (numbers at right are amino acid count, beginning with N-terminal).

((γ-α)ₘ, m = 3, n = 0, p = 1, α is a monomeric protein of the human
collagen trimerization domain 3N3F, γ is residues 19-85 of the human
ACE2 receptor with the mutations S19G, T20C, P84A, L85C)
This is the monomeric sequence γ-α.
SEQ ID NO: 1
GCIEEQAKTFLDKFNHEAEDLFYQSSLASWNYNTNITEENVQNMNNAGDKWSAFLKEQST (60)
LAQMYACGGGNLVTAFSNMDDMLQKAHLVIEGTFIYLRDSTEFFIRVRDGWKKLQLGELI (120)
PIPA (124)
((γ-α)ₘ, m = 3, n = 0, p = 1, α is a monomeric protein of the human
collagen trimerization domain 3N3F, γ is residues 19-91 of the human
ACE2 receptor with the mutations N52Q, M62K, M69K)
This is the monomeric sequence γ-α.
SEQ ID NO: 2
STIEEQAKTFLDKFNHEAEDLFYQSSLASWNYNTQITEENVQNKNNAGDKKSAFLKEQST (60)
LAQMYPLQEIQNLGGGNLVTAFSNMDDMLQKAHLVIEGTFIYLRDSTEFFIRVRDGWKKL (120)
QLGELIPIPA (130)
((γ-α)ₘ, m = 3, n = 0, p = 1, α is a monomeric protein of the human
collagen trimerization domain 3N3F, γ is residues 19-91 of the human
ACE2 receptor with the mutations N52Q, M62R, M69R)
This is the monomeric sequence γ-α.
SEQ ID NO: 3
STIEEQAKTFLDKFNHEAEDLFYQSSLASWNYNTQITEENVQNRNNAGDKRSAFLKEQST (60)
LAQMYPLQEIQNLGGGNLVTAFSNMDDMLQKAHLVIEGTFIYLRDSTEFFIRVRDGWKKL. (120)
QLGELIPIPA (130)
((α-β-γ)₃, m = 3, n = 1, m = 1, α is a monomeric protein of the 1RJ7
human trimer, β = mBSP is from the 3VT0, γ is residues 19-83 from the
ACE2 with the mutations S19G.
This is the monomeric sequence α-β-γ.
SEQ ID NO: 4
QPAVVHLQGQGSAIQVKNDLSGGVLNDWSRITMNPKVFKLHPRSGELEVLVDGTYFIYSQ (60)
VEVYYINFTDFASYEVVVDEKPFLQCTRSIETGKTNYNTCYTAGVCLLKARQKIAVKMVH (120)
ADISINMSKHTTFFGAIRLGEAPAPGAVVCVESEIRGDVTIGPRTVIHPKARIIAEAGPI (180)
VIGEGNLIEEQALIINAYPDNIKPMIIGTNNVFEVGCYSQAMKMGDNNVIESKAYVGRNV (240)
ILTSGCIIGACCNLNTFEVIPSTIEEQAKTFLDKFNHEAEDLFYQSSLASWNYNTNITEE (300)
NVQNMNNAGDKWSAFLKEQSTLAQMYG (327)
((γ-β₄-α)₃, m = 3, n = 4, m = 1, α is a monomeric protein of the 1RJ7
human trimer, β = mBSP is the 3VT0, γ from residues 19-83 of the ACE2.
This is the monomeric sequence γ-β₄-α.
SEQ ID NO: 5
STIEEQAKTFLDKFNHEAEDLFYQSSLASWNYNTNITEENVQNMNNAGDKWSAFLKEQST (60)
LAQMYGGYPDNIKPMIIGTNNVFEVGCYSQAMKMGDNNVIESKAYVGRNVILTSGCIIGA (120)
CCNLNTFEVIPENRTVIHPKARIIAEAGPIVIGEGNLIEEQALIINAYPDNIKPMIIGTN (180)
NVFEVGCYSQAMKMGDNNVIESKAYVGRNVILTSGCIIGACCNLNTFEVIPENRTVIHPK (240)
ARIIAEAGPIVIGEGNLIEEQALIINAYPDNIKPMIIGTNNVFEVGCYSQAMKMGDNNVI (300)
ESKAYVGRNVILTSGCIIGACCNLNTFEVIPENRTVIHPKARIIAEAGPIVIGEGNLIEE (360)
QALIINAYPDNIKPMIIGTNNVFEVGCYSQAMKMGDNNVIESKAYVGRNVILTSGCIIGA (420)
CCNLNTFEVIPENRTVIHPKARIIAEAGPIVIGEGNLIEEQALIINAYPDNQPAVVHLQG (480)
QGSAIQVKNDLSGGVLNDWSRITMNPKVFKLHPRSGELEVLVDGTYFIYSQVEVYYINFT (540)
DFASYEVVVDEKPFLQCTRSIETGKTNYNTCYTAGVCLLKARQKIAVKMVHADISINMSK (600)
HTTFFGAIRLGEAP (614)
EXEMPLARY PBD SEQUENCES
(One PBD in γₚ, p = 2, from ACE2 N-terminus HTH, residues 19-83
of ACE2)
SEQ ID NO: 6
STIEEQAKTFLDKFNHEAEDLFYQSSLASWNYNTNITEENVQNMNNAGDKWSAFLKEQST (60)
LAQMYP (66)
(One PBD in γₚ, p = 1, from ACE2 N-terminus HTH, residues 19-91
of ACE2)
SEQ ID NO: 7
STIEEQAKTFLDKFNHEAEDLFYQSSLASWNYNTNITEENVQNMNNAGDKWSAFLKEQST (60)
LAQMYPLQEIQNLG (74)
EXEMPLARY mBSP SEQUENCES
(mBSP from dynactin p27 BSP, 3VT0)
SEQ ID NO: 8
GAVVCVESEIRGDVTIGPRTVIHPKARIIAEAGPIVIGEGNLIEEQALIINAYPDNIKPM (60)
IIGTNNVFEVGCYSQAMKMGDNNVIESKAYVGRNVILTSGCIIGACCNLNTFEVIPEP (118)

### EXEMPLARY SYMMETRIC MULTIMERIC PROTEIN SEQUENCES

(α monomeric protein from om human collagen trimerization domain, m = 3,
3N3F)
SEQ ID NO: 9
NLVTAFSNMDDMLQKAHLVIEGTFIYLRDSTEFFIRVRDGWKKLQLGELIPIPA (54)
(α monomeric protein from αₘ human growth factor EDA-A1, m = 3, 1RJ7)
SEQ ID NO: 10
GSHMGPSGAADKAGTRENQPAVVHLQGQGSAIQVKNDLSGGVLNDWSRITMNPKVFKLHP (60)
RSGELEVLVDGTYFIYSQVEVYYINFTDFASYEVVVDEKPFLQCTRSIETGKTNYNTCYT. (120)
AGVCLLKARQKIAVKMVHADISINMSKHTTFFGAIRLGEAPAS (163)
(α monomeric protein from αₘ human langerin, m = 3, 3KQG)
SEQ ID NO: 11
ASTLNAQIPELKSDLEKASALNTKIRALQGSLENMSKLLKRQNDILQVVSQGWKYFKGNF (60)
YYFSLIPKTWYSAEQFCVSRNSHLTSVTSESEQEFLYKTAGGLIYWIGLTKAGMEGDWSW (120)
VDDTPFNKVQSARFWIPGEPNNAGNNEHCGNIKAPSLQAWNDAPCDKTFLFICKRPYVPS (180)
EP (182)
(α monomeric protein from αₘ human tetrameric diubiquitin, m = 4, 2XEW)
SEQ ID NO: 12
MQIFVKTLTGKTITLEVEPSDTIENVKAKIQDKEGIPPDQQRLIFAGKQLEDGRTLSDYN (60)
IQKESTLHLVLRLRGG (76)

### EXAMPLES

### Example 1

We have developed antibody replacements for viral neutralization from modular domain designs of proteins into multimeric proteins as antibody substitutes, extending work the inventors have previously demonstrated skill in developing for basic science⁶⁴⁻⁷⁰ and for applications⁵³. The invention is to identify human symmetric multimeric protein complexes (αₘ) such as the human collagen trimerization domain (SEQ ID NO: 9 and PDB code 3N3F, M=3), with an example shown in FIG. 2, modify these in a genetic construct by fusion to a number of n fused copies of human modified beta solenoid proteins (mBSP) such as arising from the dynactin p27 domain (SEQ ID NO: 8 modified from 3VT0) to provide length as needed for matching the geometry of the (VEP)ₘ complexes to optimize binding, and at the end attach a Pathogen binding domain (PBD) such as the N-terminus helix-turn-helix motif from the ACE2 receptor (SEQ ID NO: 6 and SEQ ID NO: 7) that the SARS-CoV-2 complex binds to (residues 19-83). The data attached herein speaks to the potency of the HTH binding (see FIG. 3).

As example 1, we consider the multimeric protein complex as antibody substitute design of SEQ ID 1 and FIG. 2, in which the human collagen trimerization domain of SEQ ID NO: 9 is modified by fusion to the n-terminal HTH domain of ACE2. As illustrated in FIG. 6, this domain can in principle bind to three RBDs of a single spike complex in the up configuration, thus providing potent neutralization capability. Because a single domain can bind with dissociation constant in the nanomolar range, per. FIG. 3, and because the dissociation constant

$${K}_{D} ∼ exp ( \frac{m Δ {G}_{B}}{{k}_{B} T} ) ,$$

where m is the number of binders (identical to the multimeric number m in this example) and ΔG_{B} is the (negative) binding free energy associated with a single binding event, the dissociation constant of the trimeric antibody substitute of SEQ ID NOS: 1-4 is likely to be in the femtomolar regime when all three RBDs are simultaneously bound by a single trimer. This avidity concept is borne out by considerable theoretical evidence and argument⁷¹⁻⁷⁵ and by recent evidence of engineered nanobody binding to the down domain of the spike protein where trimers have likely femtomolar affinity⁷⁶. FIG. 4 illustrates several relevant points from simulations supporting this. First, FIG. 4A shows that several of the most concerning variants (alpha, beta, gamma) have little variation in interfacial hydrogen bound count measured in >10 ns of YASARA⁶³ all atom molecular dynamics simulations of the PBD from the N-terminus (SEQ ID NO: 6 or SEQ ID NO: 7) binding to the RBD. The mutations produce small variation of interfacial hydrogen bond number on average. We find an excellent correlation of the hydrogen bond count at the protein-protein interface with the binding energy measured in the MM/GBSA program HawkDock⁷⁷. A separate correlation of binding energy point mutations of the RBD binding to the HTH shows that the relation between the estimated HawkDock change in binding energy with mutation and the measured Deep Mutational Scan energies is given by (all in kcal/mole)

ΔΔG_{Mut-DMS}=0.03437*(ΔΔG_{Mut-HawkDock}−3.8)

from which we can predict, with the theoretical ΔΔG_{Mut-HawkDock}, the dissociation constants relative to wildtype (WT) as

$$\frac{{K}_{D}}{{K}_{D , WT}} = exp ( 1000 * 4.18 * \frac{Δ Δ {G}_{M u t - D M S}}{R T} )$$

where T is taken at room temperature and R=8.29 J/mole-K is the ideal gas constant. This is the third column of FIG. 4A. These results support the independence of the PBD binding strength with respect to Spike RBD variant sequence since they vary less than a factor of 2 from wild type. FIG. 4B shows a monomeric protein of SEQ ID NO: 3 bound to the RBD. FIG. 4C shows simulation results for 12 ns of simulation time of a trimer of SEQ ID NO: 3 bound to the RBDs of the 7KMS PDB file, constrained to the positions of 7KMS to mimic the full spike trimer. We find >3X the number of interfacial hydrogen bonds on average compared to the simulation of a single monomeric protein of HTH bound to a single RBD. This strongly supports the avidity concept above, and (i) the linear relationship of binding energy with number of bound interfaces, and (ii) the linear relationship of binding energy with interfacial hydrogen bound counts we have observed, we expect an affinity of the trimer of as low as femtomolar.

By working with human constructs for the multimeric proteins as antibody substitutes containing a minor number (≤4-5) of point mutations, it is anticipated that our modular protein designs will be resistant to immunogenic response/rejection, and proteolytic degradation inside the body. Moreover, by using common domains from ubiquitous human proteins, the potential for autoimmune response is avoided.

By choosing protein domains for our multimeric proteins as antibody substitutes which have been expressed from well proven, scalable, industrialized prokaryotic or single celled eukaryotic processes, we save the expense and unpredictability of monoclonal antibody production. FIG. 5 shows successful expression of trimers from SEQ ID NO: 2 and SEQ ID NO: 3.

The molecular weight of each monomeric protein in our multimeric protein complex as antibody substitute designs is relatively small compared to antibodies. The design of SEQ ID 1 and FIG. 2 has a mass of approximately 14 KDa per binding unit, compared with 150KDa typical of monoclonal antibodies (mABs). The significance of this is that less protein mass is needed per prophylactic or therapeutic unit to achieve the same efficacy as for mABs.

### Example 2

In example 2, we consider the somewhat larger construct of FIG. 6, and SEQ ID NO: 4 where the modified multimeric protein am is taken from the trimeric EDA-1 complex (SEQ ID NO: 10 and PDB Code 1RJ7), and we add a single β=mBSP element from the dynactin p27 complex (SEQ ID NO:8 and PDB Code 3VT0), and the γ=HTH domain from the ACE2 protein as mentioned above. This complex has a mass of approximately 42 KDa.

This design can access the RBD domain even when it is flipped out away from the spike complex, and thus is amenable to a wider range of binding conformations than the smaller design of FIG. 2, because the center to center spacing of the HTH regions is about 7-8 nm, comparable to the RBD separation when all are fully out of the spike complex.

### Example 3

The much larger multimeric protein complex as antibody substitute design of FIG. 7 and SEQ ID NO: 5 is made from the modified am EDA-1 complex (SEQ ID NO: 10) as per EXAMPLE 2, and this is fused to a fused 4-fold repeat of the β=mBSP complex modified from the human dynactin p27 protein (SEQ ID NO: 8), which is attached at the end to γ=HTH PBD from the ACE2 protein (SEQ ID NO: 7). The choice of four mBSP repeats provides an HTH-to-HTH separation of approximately 24 nm. The spike complexes on the surface of the SARS-CoV-2 virus are separated on average about 22 nm⁷⁶, so this flexible trimer could potentially neutralize three spike proteins at once. The mass per fused monomeric protein is approximately 72 KDa, but if successful in neutralizing three spike proteins at once, the mass per RBD is like that of FIG. 2 and SEQ ID NO: 1.

### Example 4

The HTH complex itself, in dimer form, can be a potent multimeric protein complex as antibody substitute neutralizing agent. The dimer construct of FIG. 8 and SEQ ID NO: 6 and SEQ ID NO: 7 has been demonstrated to have negligible affinity difference from the full length ACE2 per the inventors' data of FIG. 3. The mass per binding unit here is the smallest (about 7 KDa) of any of the other constructs, and we estimate the dimer self-affinity to be K_{D}≈250 nM, weaker than the affinity of the HTH to the RBD domain.

This multimeric protein complex as antibody substitute complex can be mutated by up to 4 amino acids to achieve higher affinity binding with the RBD without inducing immunogenic response, and such mutations can enhance the affinity per HTH to sub-nanomolar KD values⁷⁸.

If necessary, particularly in attachment to the complexes of EXAMPLES 1-3, we can modify the hydrophobic “underside” of the HTH complex by 1-4 residues to prevent self-association.

### Example 5

By fusing a specific human serum albumin binding sequence to the side of the trimers in the multimeric protein complex as antibody substitutes discussed in EXAMPLES 1-3 opposite the binding face to the spike proteins, we can attach to albumin in the blood. For example, the SA2 peptide invented by Genentech⁷⁹⁻⁸², has specific binding to serum albumin. This provides steric hindrance to viral binding in addition to the explicit blocking of the spike proteins and engenders enhanced lifetimes in vivo.

### Example 6

The multimeric protein complex as antibody substitute inventions herein, while using specific examples of binding to the SARS-CoV-2 virus, are not solely restricted to this application. For example, the general multimeric protein complex as antibody substitute schema (α-βₙ-γₚ)ₘ or (γₚ-βₙ-α)ₘ can be extended to develop neutralization agents for the trimeric haemagglutinin VEPs on the surface of influenza virions, the tetrameric neuraminidase complexes on influenza virions, or the trimeric gp120 VEPs on the surface of HIV virions.

Additionally, the general (α-βₙ-γₚ)ₘ or (γₚ-βₙ-α)ₘ scheme can be extended to binding to multimeric fusion complexes on microorganisms or tumor cells.

### REFERENCES

1. M.-Y. Chou, C.-Y. Fan, C.-C. Huang and H.-C. Li, U.S. Pat. No. 10,183,986B2 (2019).
2. P. Bruenker, C. F. Koller, S. Grau-Richards, E. Moessner and P. Umana, U.S. Pat. No. 9,975,958B2 (2018).
3. V. J. Sanchez-Arevalo Lobo, A. M. Cuesta, L. Sanz, M. Compte, P. Garcia, J. Prieto, F. J. Blanco and L. Alvarez-Vallina, International Journal of Cancer 119 (2), 455-462 (2006).
4. A. M. Cuesta, D. Sanchez-Martin, A. Blanco-Toribio, M. Villate, K. Enciso-Alvarez, A. Alvarez-Cienfuegos, N. Sainz-Pastor, L. Sanz, F. J. Blanco and L. Alvarez-Vallina, Mabs 4 (2), 226-232 (2012).
5. A. M. Cuesta, N. Sainz-Pastor, J. Bonet, B. Oliva and L. Alvarez-Vallina, Trends in Biotechnology 28 (7), 355-362 (2010).
6. C. B. Shoemaker and H. Feng, (2015).
7. J. A. Wirz, S. P. Boudko, T. F. Lerch, M. S. Chapman and H. P. Baechinger, Matrix Biology 30 (1), 9-15 (2011).
8. S. G. Hymowitz, D. M. Compaan, M. H. Yan, H. J. A. Wallweber, V. M. Dixit, M. A. Starovasnik and A. M. de Vos, Structure 11 (12), 1513-1520 (2003).
9. S. G. Hymowitz, D. M. Compaan, M. Yan, H. Ackerly, V. M. Dixit, M. A. Starovasnik and A. M. De Vos, Worldwide Protein Data Bank (2003).

10. H. Feinberg, A. S. Powlesland, M. E. Taylor and W. I. Weis, Journal of Biological Chemistry 285 (17), 13285-13293 (2010).
11. H. Feinberg, A. S. Powlesland, M. E. Taylor and W. I. Weis, Worldwide Protein Data Bank (2010).
12. A. Bremm, S. M. V. Freund and D. Komander, Worldwide Protein Data Bank (2010).
13. A. Bremm, S. M. V. Freund and D. Komander, Nature Structural & Molecular Biology 17 (8), 939-U947 (2010).
14. Z. S. Derewenda, U. Derewenda and A. Kowalska, Worldwide Protein Data Bank (2012).
15. K. Kuhnel, S. Veltel, I. Schlichting and A. Wittinghofer, Structure 14 (2), 367-378 (2006).
16. K. Kuhnel, S. Veltel, I. Schlichting and A. Wittinghofer, Worldwide Protein Data Bank (2006).
17. J. Lan, J. Ge, J. Yu, S. Shan, H. Zhou, S. Fan, Q. Zhang, X. Shi, Q. Wang, L. Zhang and X. Wang, Nature (2020).
18. X. Wang, J. Lan, J. Ge, J. Yu and S. Shan, Worldwide Protein Data Bank (2020).
19. L. A. Graham, W. S. Qin, S. C. Lougheed, P. L. Davies and V. K. Walker, Journal of Molecular Evolution 64 (4), 387-398 (2007).
20. S. P. Graether and B. D. Sykes, European Journal of Biochemistry 271 (16), 3285-3296 (2004).
21. E. K. Leinala, P. L. Davies and Z. C. Jia, Structure 10 (5), 619-627 (2002).
22. Y. C. Liou, A. Tocilj, P. L. Davies and Z. C. Jia, Nature 406 (6793), 322-324 (2000).
23. Y. F. Mok, F. H. Lin, L. A. Graham, Y. Celik, I. Braslaysky and P. L. Davies, Biochemistry 49 (11), 2593-2603 (2010).
24. B. L. Pentelute, Z. P. Gates, V. Tereshko, J. L. Dashnau, J. M. Vanderkooi, A. A. Kossiakoff and S. B. H. Kent, J. Am. Chem. Soc. 130 (30), 9695-9701 (2008).
25. K. J. Lauersen, A. Brown, A. Middleton, P. L. Davies and V. K. Walker, Cryobiology 62 (3), 194-201 (2011).
26. A. J. Middleton, A. M. Brown, P. L. Davies and V. K. Walker, Febs Letters 583 (4), 815-819 (2009).
27. S. L. Roderick, Worldwide Protein Data Bank (1995).
28. K. Brown, F. Pompeo, S. Dixon, D. Mengin-Lecreulx, C. Cambillau and Y. Bourne, Worldwide Protein Data Bank (2000).
29. D. Kostrewa, A. D'Arcy and M. Kamber, Worldwide Protein Data Bank (2001).
30. L. R. Olsen and S. L. Roderick, Worldwide Protein Data Bank (2001).
31. B. I. Lee and S. W. Suh, Worldwide Protein Data Bank (2004).
32. V. E. Pye, A. P. Tingey, R. L. Robson and P. C. E. Moody, Worldwide Protein Data Bank (2004).
33. C. Kisker, H. Schindelin and D. C. Rees, Worldwide Protein Data Bank (1996).
34. T. W. Beaman, K. W. Vogel, D. G. Drueckhammer, J. S. Blanchard and S. L. Roderick, Worldwide Protein Data Bank (2002).
35. L. E. Kehoe, J. Snidwongse, P. Courvalin, J. B. Rafferty and I. A. Murray, Worldwide Protein Data Bank (2003).
36. L. R. Olsen, B. Huang, M. W. Vetting and S. L. Roderick, Worldwide Protein Data Bank (2004).
37. H. J. Lee, B. Rakic, M. Gilbert, W. W. Wakarchuk, S. G. Withers and N. C. J. Strynadka, Worldwide Protein Data Bank (2009).
38. S. M. Anderson, Z. Wawrzak, 0. Onopriyenko, S. N. Peterson, W. F. Anderson, A. Savchenko and D. Center for Structural Genomics of Infectious, Worldwide Protein Data Bank (2011).
39. X. G. Wang, L. R. Olsen and S. L. Roderick, Worldwide Protein Data Bank (2002).
40. C. M. Baffling and C. R. H. Raetz, Worldwide Protein Data Bank (2009).
41. S. Kumar and S. Gourinath, Worldwide Protein Data Bank (2011).
42. G. Kozlov, K. Gehring and I. Montreal-Kingston Bacterial Structural Genomics, Worldwide Protein Data Bank (2008).
43. Y. Kim, N. Maltseva, L. Papazisi, W. Anderson and A. Joachimiak, Worldwide Protein Data Bank (2009).
44. A. Dickmanns, S. Damerow, P. Neumann, E. C. Schulz, A. Lamerz, F. Routier and R. Ficner, Worldwide Protein Data Bank (2010).
45. M. J. Morrison and B. Imperiali, Worldwide Protein Data Bank (2013).
46. A. Masoudi, C. R. H. Raetz and C. W. Pemble, Worldwide Protein Data Bank (2013).
47. J. Benach, Chen, Y., Vorobiev, S. M., Seetharaman, J., Ho, C. K., Janjua, H., Conover, K., Ma, L-C., Xiao, R., et al. , (2014).
48. J. Benach, Y. Chen, S. M. Vorobiev, J. Seetharaman, C. K. Ho, H. Janjua, K. Conover, L. C. Ma, R. Xiao, T. B. Acton, G. T. Montelione, J. F. Hunt, L. Tong and C. Northeast Structural Genomics, Worldwide Protein Data Bank (2007).
49. S. S. Hegde, M. W. Vetting, S. L. Roderick, L. A. Mitchenall, A. Maxwell, H. E. Takiff and J. S. Blanchard, Worldwide Protein Data Bank (2005).
50. M. A. Kennedy, S. Ni, G. W. Buchko and H. Robinson, Worldwide Protein Data Bank (2006).
51. M. A. Kennedy, S. Ni and G. M. Sheldrick, Worldwide Protein Data Bank (2008).
52. T. W. Beaman, M. Sugantino and S. L. Roderick, Worldwide Protein Data Bank (1998).
53. G.-y. L. Daniel Cox, Michael Toney, Xi Chen, Josh Hihath, Gergely Zimanyi, Natha Robert Hayre,Ma ria Peralta U.S. Pat. No. 10,287,332B2 (2019).
54. T. F. Smith and M. S. Waterman, Journal of Molecular Biology 147 (1), 195-197 (1981).
55. S. B. Needleman and C. D. Wunsch, Journal of Molecular Biology 48 (3), 443-+(1970).
56. W. R. Pearson and D. J. Lipman, Proceedings of the National Academy of Sciences of the United States of America 85 (8), 2444-2448 (1988).
57. S. F. Altschul, W. Gish, W. Miller, E. W. Myers and D. J. Lipman, Journal of Molecular Biology 215 (3), 403-410 (1990).
58. S. F. Altschul, T. L. Madden, A. A. Schaffer, J. H. Zhang, Z. Zhang, W. Miller and D. J. Lipman, Nucleic Acids Research 25 (17), 3389-3402 (1997).
59. S. Henikoff and J. G. Henikoff, Proceedings of the National Academy of Sciences of the United States of America 89 (22), 10915-10919 (1992).
60. S. Karlin and S. F. Altschul, Proceedings of the National Academy of Sciences of the United States of America 90 (12), 5873-5877 (1993).
61. S. Karlin and S. F. Altschul, Proceedings of the National Academy of Sciences of the United States of America 87 (6), 2264-2268 (1990).
62. A. Basit, T. Ali and S. U. Rehman, Journal of Biomolecular Structure & Dynamics (2020).
63. E. Krieger and G. Vriend, Bioinformatics 30, 2981-2982 (2014).
64. M. D. R. Peralta, A. Karsai, A. Ngo, C. Sierra, K. T. Fong, N. R. Hayre, N. Mirzaee, K. M. Ravikumar, A. J. Kluber, X. Chen, G.-y. Liu, M. D. Toney, R. R. Singh and D. L. Cox, Acs Nano 9 (1), 449-463 (2015).
65. L. P. Heinz, K. M. Ravikumar and D. L. Cox, Nano Letters 15 (5), 3035-3040 (2015).
66. Z. Peng, A. S. Parker, M. D. R. Peralta, K. M. Ravikumar, D. L. Cox and M. D. Toney, Biophysical Journal 113 (9), 1945-1955 (2017).
67. Z. Peng, M. D. R. Peralta and M. D. Toney, Biochemistry 56 (45), 6041-6050 (2017).
68. A. S. Parker, K. M. Ravikumar and D. L. Cox, Soft Matter 13, 6218-6226 (2017).
69. R. A. Baarda, T. L. Marianchuk, M. D. Toney and D. L. Cox, Soft Matter 14 (40), 8095-8104 (2018).
70. Z. Peng, M. D. R. Peralta, D. L. Cox and M. D. Toney, PloS one 15 (2), e0229319-e0229319 (2020).
71. P. I. Kitov and D. R. Bundle, Journal of the American Chemical Society 125 (52), 16271-16284 (2003).
72. G. Vauquelin, G. Bricca and I. Van Liefde, Fundamental & Clinical Pharmacology 28 (5), 530-543 (2014).
73. G. Vauquelin and S. J. Charlton, British Journal of Pharmacology 168 (8), 1771-1785 (2013).
74. S. J. Charlton and G. Vauquelin, British Journal of Pharmacology 161 (6), 1250-1265 (2010).
75. G. Vauquelin and S. J. Charlton, British Journal of Pharmacology 161 (3), 488-508 (2010).
76. M. Schoof, B. Faust, R. A. Saunders, S. Sangwan, V. V. Rezelj, N. Hoppe, M. Boone, C. Billesboelle, M. Zimanyi and I. Deshpande, bioRxiv (2020).
77. G. Weng, E. Wang, Z. Wang et al., Nucleic Acids Research 47, Supplement W, W322-W330 (2019).
78. S. Klein, M. Cortese, S. L. Winter, M. Wachsmuth-Melm, C. J. Neufeldt, B. Cerikan, M. L. Stanifer, S. Boulant, R. Bartenschlager and P. Chlanda, BioRxiv (2020).
79. K. K. Chan, D. Dorosky, P. Sharma, S. A. Abbasi, J. M. Dye, D. M. Kranz, A. S. Herbert and E. Procko, Science (New York, N.Y.) (2020).
80. M. S. Dennis, H. Jin, D. Dugger, R. Yang, L. McFarland, A. Ogasawara, S. Williams, M. J. Cole, S. Ross and R. Schwall, Cancer Research 67 (1), 254-261 (2007).
81. M. S. Dennis, M. Zhang, Y. G. Meng, M. Kadkhodayan, D. Kirchhofer, D. Combs and L. A. Damico, Journal of Biological Chemistry 277 (38), 35035-35043 (2002).
82. M. S. Dennis, Patent No. US2010104588-A1.
83. M. S. Dennis, Patent No. US2007202045-A1.

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

1. A multimeric protein complex as antibody substitute complex comprising a plurality (e.g., m≥2) of monomeric proteins with modular protein domains of the form (α-βₙ-γₚ)ₘ or (γₚ-βₙ-α)ₘ wherein the monomeric proteins α-βₙ-γₚ or γₚ-βₙ-α comprise fused protein domains wherein:
α is a monomeric protein from a symmetric human multimeric protein complex of point group symmetry Cₘ or Dₘ,
β is a fused domain of n modified beta solenoid proteins (mBSPs) with n≥0, and
γₚ is a complex of p pathogen binding domains (PBDs) either fused or bound to each other by intermolecular forces and wherein p≥1.

2. The multimeric protein complex as antibody substitute of claim 1, wherein the multimeric protein complex as antibody substitute is symmetrical.

3. The multimeric protein complex as antibody substitute of claim 2, wherein the multimeric protein complex as antibody substitute has two-fold symmetry, three-fold symmetry, four-fold symmetry, five-fold symmetry, six-fold symmetry, or twelve-fold symmetry.

4-8. (canceled)

9. The multimeric protein complex as antibody substitute of claim 1, wherein the modular protein domain a is a monomeric protein from a wild type symmetric human multimeric protein complex αₘ.

10. (canceled)

11. The multimeric protein complex as antibody substitute of claim 1, wherein α is a monomeric protein from:
(a) the m=3 human collagen trimerization domain which is at least 90%, 95%, 98%, or 99% identical to SEQ ID NO: 9. or
(b) the m=3 human growth factor EDA-Al which is at least 90%, 95%, 98%, or 99% identical to SEQ ID NO: 10; or
(c) the m=3 human Langerin which is at least 90%, 95%, 98%, or 99% identical to SEQ ID NO: 11; or
(d) the m=4 human tetrameric diubiquitin which is at least 90%, 95%, 98%, or 99% identical to SEQ ID NO: 12.

12-14. (canceled)

15. The multimeric protein complex as antibody substitute of claim 1, where n=0, p=1 and the protein binding domain (PBD) γ is at least 90%, 95%, 98% or 99% identical to the N-terminus domain (residues 19-85 or residues 19-91) of the human ACE2 receptor protein of SEQ ID NO: 6 or SEQ ID NO: 7.

16. The multimeric protein complex as antibody substitute of claims 1, wherein the monomeric protein sequence is at least 90%, 95%, 98% or 99% identical to SEQ ID NO:1, 2 or 3.

17. (canceled)

18. (canceled)

19. The multimeric protein complex as antibody substitute of claim 1, wherein the modified human beta solenoid (mBSP) is: (a) at least 80%, 90%, 95%, 98%, or 99% identical to the dynactin p27 domain (3VTO) of SEQ ID NO: 8. or (b) modified to be at least 80%, 90%, 95%, 98% or 99% identical to the human Retinitis Pigmentosa Protein 2 (RP2) (2BX6).

20. The multimeric protein complex as antibody substitute of claim 1, wherein the monomeric sequence is at least 90%, 95%, 98% or 99% identical to SEQ ID NO: 4 and n=1 and p=1, or to SEQ ID NO: 5, and n=4 and p=1.

21. (canceled)

22. The multimeric protein complex as antibody substitute of claim 1, wherein the sequence is of the form y2 and y is at least 90%, 95%, 98% or 99% identical to SEQ ID NO: 6 or SEQ ID NO: 7.

23. (canceled)

24. The multimeric protein complex as antibody substitute of claim 1, wherein the pathogen binding domain is at least 90%, 95%, 98% or 99% identical to the helix-turn-helix (HTH) complex from the N-terminus of the ACE2 receptor protein of SEQ ID NO: 6 or SEQ ID NO: 7.

25. The multimeric protein complex as antibody substitute of claim 1, wherein at least one (e.g., 1, 2, 3, 4, 5, or more) amino acid of the a or modified human beta solenoid domain is modified to allow attachment to a nanoparticle, a solid support, or other biological molecule.

26. The multimeric protein complex as antibody substitute of claim 1, wherein the multimeric protein complex is attached to a nanoparticle, a solid support, or other biological molecule.

27. The multimeric protein complex as antibody substitute of claim 1, wherein the multimeric protein complex is attached to human serum albumin.

28. The pathogen binding domain of the multimeric protein complex as antibody substitute of claim 1, wherein at least one (e.g., 1, 2, 3, 4, or more) amino acid of one or more of the module domains is modified to allow attachment to a nanoparticle, a solid support, or other biological molecule.

29. A multimeric protein complex as antibody substitute comprising a plurality of pathogen binding domains (e.g., 2, 3, 4, or more).

30. The multimeric protein complex as antibody substitute of claim 29, wherein the pathogen binding domain is modified to be at least 90%, 95%, 98% or 99% identical to the HTH domain (residues 19-85 or 19-91) of the N-terminus of the ACE2 receptor protein of SEQ ID NO: 6 or SEQ ID NO: 7.

31. The multimeric protein complex as antibody substitute of claim 30, wherein at least one (e.g., 1, 2, 3, 4, or more) amino acid of either or all pathogen binding domains are modified to allow attachment to a nanoparticle, a solid support, or other biological molecule.

32. A method for neutralizing a pathogen comprising contacting said pathogen with the multimeric protein complex as antibody substitute of claim 1, wherein one or more pathogen binding domains binds to one or more sites on the pathogen.

33. A method for immobilizing a pathogen comprising contacting said pathogen with the multimeric protein complex as antibody substitute of claim 1, wherein one or more pathogen binding domains binds to one or more sites on the pathogen.

34. (canceled)
